(19) United States
(12) Patent Application Publication
(10) Pub. No.: US 2016/0361385 A1
(43) Pub. Date: Dec. 15, 2016

(54) METHOD FOR TREATING ALZHEIMER'S DISEASE USING SUPERIOR STABILITY TUBULIN CONSRUCTS

(71) Applicants: Jack A. Tuszynski, Edmonton (CA); Nancy Woolf, Las Vegas, NV (US)

(72) Inventors: Jack A. Tuszynski, Edmonton (CA); Nancy Woolf, Las Vegas, NV (US)

(21) Appl. No.: 15/180,764

(22) Filed: Jun. 13, 2016

Related U.S. Application Data
(60) Provisional application No. 62/174,042, filed on Jun. 11, 2015.

(51) Int. Cl. A61K 38/17

(52) U.S. Cl. CPC A61K 38/1709; A61K 48/00

(57) ABSTRACT

A method of providing a therapeutic benefit to a patient for Alzheimer's Disease (AD) is disclosed. A superior stabilizing-tubulin (SS-tubulin) is administered, either directly or indirectly through gene therapy. Mutated alpha SS-tubulins and mutated beta SS-tubulins are disclosed for use in the method.

### CROSS-REFERENCE TO RELATED APPLICATIONS

This application claims priority to and is a non-provisional of U.S. Patent Application Ser. No. 62/174,042 (filed Jun. 11, 2015) the entirety of which is incorporated herein by reference.

### REFERENCE TO A SEQUENCE LISTING

This application refers to a “Sequence Listing” listed below, which is provided as an electronic document entitled “TUSZ17282_ST25” (8 kb created on Jun. 6, 2016) which is incorporated herein by reference in its entirety.

### BACKGROUND OF THE INVENTION

The subject matter disclosed herein relates to methods of treating diseases and to methods of treating Alzheimer's disease in particular. According to the most recent update by the Alzheimer's Association, Alzheimer's disease (AD) affects over 5 million Americans and the number of cases is expected to rise to over 13 million by year 2050. In the U.S., AD has risen to the sixth leading cause of death and is associated with societal and economic costs estimated at $214 billion a year. The World Alzheimer Report 2014 estimates some 36 million persons have AD worldwide and by 2050 there may be as many as 135 million afflicted, with costs escalating into $trillions (calculated in U.S. dollars). These alarming numbers underscore the urgent need to find an effective treatment to prevent the disorder from progressing, ideally before symptoms even appear.

The hallmark symptom of AD is dementia—a loss of memory and a deficit in thinking skills needed to function successfully in everyday life. Effectively treating this “great thief of quality-of-life” stubbornly eludes the scientific community. Currently there are five FDA-approved drugs to treat AD, none of which is highly effective in treating cognitive impairment; moreover, hundreds of clinical trials for AD patients fail to find a treatment that effectively treats AD-related dementia.

One logical strategy is to attack the neuropathology that forms in AD brain with the goal of preventing, halting, or even reversing progression. AD brain is characterized by an abundance of neurofibrillary tangles and senile plaques, two forms of neural debris that accumulate in particular brain regions. The density of tangles and plaques correlates positively with stage of dementia. Tangles and plaque are made of abnormal protein aggregates that are stubbornly insoluble, unlike their normal constituent proteins that undergo continuous breakdown and replenishment. The reason these insoluble aggregates accumulate in particular parts of AD brain eludes our complete understanding; however, one clue is that the density of plaques and tangles is typically greatest in hippocampus, parahippocampal regions, parts of the amygdala, and select regions of cerebra 1 cortex-precisely those brain regions that exhibit the greatest levels of neuroplasticity (i.e. change in structural and functional connectivity). Microtubules are one of the primary mediators of neuroplasticity-being responsible for structural and functional changes in connectivity related to memory and memory-deficits in AD. Thus, it follows that compromise to the microtubule system would lead to dementia.

Despite the marked presence of plaques and tangles in AD brain, it is disappointing that targeting plaques and tangles has not yet proven successful in clinical trials. Although plaques and tangles are clearly tied to dementia, recent and currently ongoing clinical trials to reverse plaque formation with monoclonal antibodies directed against the toxic amyloid-β peptide are not markedly improving cognitive status of AD patients despite promising results in preclinical studies. Immunological strategies to reduce phospho-tau (and tangle formation) and anti-tangle drugs show promise in preclinical studies but remain to be tested clinically. Nonetheless, an ever-present concern with immunotherapy is encephalitis, a sometimes realized potential side effect when using antibodies to combat plaques and tangles. For these and other reasons, we need to find better methods to prevent plaques and tangles from forming in AD brain.

Recent advances in gene therapy found their way to clinical trials for treating AD, the rationale being that a more efficient delivery system might produce better clinical results for certain promising agents. Obvious gene candidates include those for the neurotrophins—key mediators of cell growth and survival. Nerve growth factor (NGF) is a neurotrophic factor that facilitates cell survival and function of cholinergic neurons located in the basal forebrain-precisely the neurons known to degenerate in AD. Clinical trials of gene therapies for AD include both ex vivo and adeno-associated virus (AVV) vectors carrying NGF DNA deep into the brain. Despite overwhelming enthusiasm derived largely from preclinical studies, NGF gene therapies have met with limited success thus far. In the earliest phase 1 clinical trials, the rate of cognitive decline in AD patients was slowed by approximately 22 months. In a recent clinical trial examining ten AD patients receiving AAV-NGF, there was no evidence of accelerated decline in cognitive function at 2 years post-operation; and despite hope that future trials may yield promising results, it is disconcerting that the authors were not able to make a case for NGF-gene therapy even mildly improving the rate of decline as compared to historical rates of AD progression or compared to placebo groups in other AD drug trials.

Success may also be realized by indirect means of affecting microtubule stabilization. Because many of the microtubule stabilizers are anti-cancer drugs with potent side effects, toxicity is an inherent problem as are their off-target interactions. For this reason, researchers propose not trying to stabilize microtubules directly, but instead redistribute zinc away from plaques to increase intracellular zinc levels and thereby stabilize microtubules secondarily. This approach corrects zinc imbalance in AD, increasing intracellular levels of zinc levels to enhance microtubule stability. Nonetheless, it remains unclear how altering zinc levels will affect other metabolic pathways in unaffected cells. It is also a non-trivial task to direct specific zinc changes in the brain and to make sure appropriate zinc levels (not too high and not too low) are stably present inside neurons without further fueling plaque formation since β-amyloid aggregation is enhanced by zinc accumulation outside neurons.

The discussion above is merely provided for general background information and is not intended to be used as an aid in determining the scope of the claimed subject matter.

### BRIEF DESCRIPTION OF THE INVENTION

A method of providing a therapeutic benefit to a patient for Alzheimer's Disease (AD) is disclosed. A superior stabilizing-tubulin (SS-tubulin) is administered, either directly or indirectly through gene therapy. Mutated alpha SS-tubulins and mutated beta SS-tubulins are disclosed for use in the method.

In a first embodiment, a method of treating a patient to provide a therapeutic benefit for Alzheimer's Disease (AD) is provided. The method comprises administering to a human patient at least an alpha tubulin that is at least 70% homologous with SEQ ID NO. 3, wherein at least one point mutation is present, the at least one point mutation being selected from the group consisting of Asn47; Val58; Lys90; Lys218; Lys220; Gln254 or Ala254; Asn327 and combinations thereof.

In a second embodiment, a method of treating a human patient to provide a therapeutic benefit is provided. The method comprises administering at least a beta tubulin that is at least 70% homologous with SEQ ID NO. 4, wherein at least one point mutation is present, the at least one point mutation being selected from the group consisting of Glu122; Asn74 and combinations thereof.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic depiction of αβ-tubulin dimers assembling longitudinally into microtubule protofilament, which bind through lateral contacts with other protofilaments to form microtubules;

FIG. 2 is a flow diagram depicting one method for screening for mutated tubulins;

FIG. 3 depicts Table 3 showing hydrogen bond energy between alpha and beta tubulins on the longitudinal interface;

FIG. 4 depicts Table 4 showing hydrogen bond energy between adjacent alpha tubulins and between adjacent beta tubulins; and

FIG. 5 depicts Table 5 showing hydrogen bond energy between alpha and beta tubulins on the latitudinal interface.

### DETAILED DESCRIPTION OF THE INVENTION

A novel treatment strategy is provided that protects against the pathology cascade underlying AD by fortifying brain microtubules in damaged regions of an AD brain. A growing body of literature supports the contention that microtubules are a central, if not primary, issue in AD pathology. The disclosed method focuses on how microtubule proteins (tubulins) change structural conformation upon drug binding. Rational molecular drug designs allow structural alterations of targeted proteins in silica and testing of the effects of drug binding on those targets. A yet untapped corollary to that approach is to permanently alter the target protein to achieve the same optimal state that the effective drug produces only transiently. To that end, this disclosure provides a method to alter the structure of tubulin protein in order to produce more structurally stable microtubules. An enduring increase in microtubule stability significantly facilitates transport throughout the cell by shifting the dynamic state of microtubules away from excessive cytoskeleton breakdown (depolymerization). The net effect of this intervention protects the cell and prevents cellular degeneration.

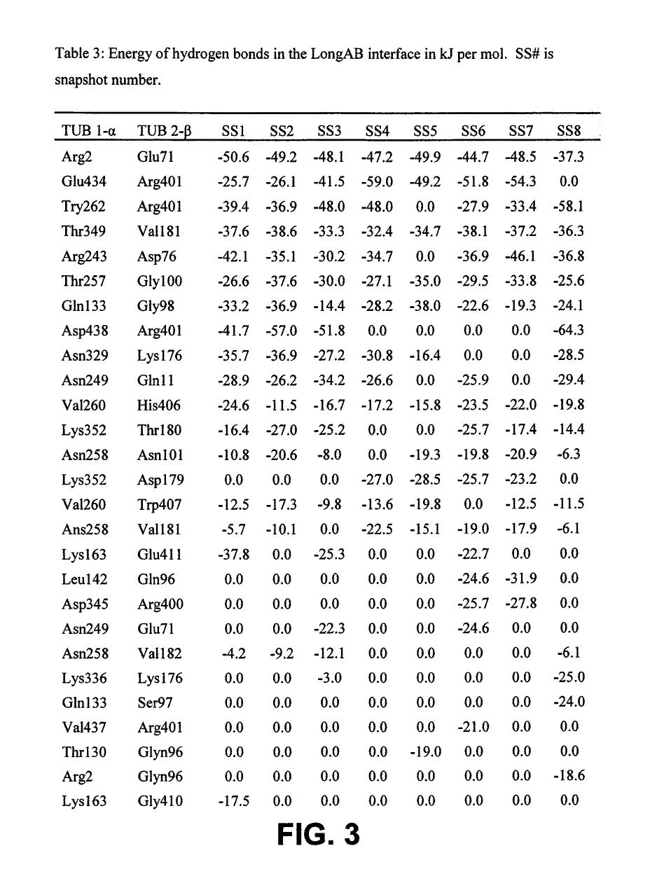

As discussed in further detail elsewhere in this specification, stabilizing point mutations to alpha tubulin are given in Table 1 while stabilizing point mutations to beta tubulin are given in Table 3 (see FIG. 3). The resulting non-naturally occurring tubulins are provided in SEQ ID NO. 3 (mutated alpha tubulin) and SEQ ID NO. 4 (mutated beta tubulin).

In one embodiment, a non-naturally occurring tubulin is administered to a patient to provide a therapeutic benefit to the patient. Modes of administration include, but are not limited to, gene therapy, stereotaxic intracerebral injection, oral or intravenous introduction of nanocapsules or nanoparticle containing or bound to the tubulin in conjunction. In some embodiment, the nanocapsules or nanoparticles are configured to localize the tubulin to a particular brain region of interest. The patient may be experiencing symptoms of AD or the patient may utilize the method to prevent or delay the onset of AD prior to experiencing symptoms. In one embodiment, gene therapy is used to administer a gene that encodes for the non-naturally occurring tubulin. The administered tubulin may be an alpha tubulin, a beta tubulin or a combination thereof.

The mutated alpha tubulin is homologous with SEQ ID NO. 3 wherein at least one of the following seven point mutations are present: αAsp47 to Asn; αAla58 to Val; αGlu90 to Lys; αAsp218 to Lys; αGlu220 to Lys; αGlu254 to Gln or Ala; αAsp327 to Asn. In one embodiment, at least two of these point mutations are present. In another embodiment, at least three of these point mutations are present. In another embodiment, at least four of these point mutations are present. In another embodiment, at least five of these point mutations are present. In another embodiment, at least six of these point mutations are present. In another embodiment, all seven of these point mutations are present. Various isotypes of alpha tubulin are known that share a relatively high degree of homology. In one embodiment, the mutated alpha tubulin has fewer than five hundred residues and is at least 70% homologous with SEQ ID NO. 3. In one such embodiment, the tubulin is at least 80% homologous with SEQ ID NO. 3. In one such embodiment, the tubulin is at least 90% homologous with SEQ ID NO. 3. In one such embodiment, the tubulin is at least 95% homologous with SEQ ID NO. 3.

The mutated beta tubulin is homologous with SEQ ID NO. 4 wherein at least one of the following two point mutations are present: βLys122 to Glu; βAsp74 to Asn. In one embodiment, both of these point mutations are present. According to the numbering scheme used by RCSB PDB the aforementioned residues are labeled as βLys124 and βAsp76, respectively. Various isotypes of beta tubulin are known that share a relatively high degree of homology. In one embodiment, the mutated beta tubulin has fewer than five hundred residues and is at least 70% homologous with SEQ ID NO. 4. In one such embodiment, the tubulin is at least 80% homologous with SEQ ID NO. 4. In one such embodiment, the tubulin is at least 90% homologous with SEQ ID NO. 4. In one such embodiment, the tubulin is at least 95% homologous with SEQ ID NO. 4.

A more detailed discussion is provided below.

Stabilizing microtubules in vulnerable brain cells of AD patients (who are either symptomatic or asymptomatic) has potential to prevent, halt, or reverse the formation of tau-tangles and possibly plaques. The range of external parameters, such as ion concentrations that optimally stabilize microtubules, has a narrow window. A critical balance must exist between stability and instability to maintain proper microtubule function, since both over-stabilized and under-stabilized microtubules lead to cell death. Nonetheless, preclinical data indicate that increasing stability of microtubules does improve cognition and prevent neuropathology. Drugs that stabilize microtubules by inhibiting depolymerization reduce cognitive deficits and AD-like pathology in aged tau transgenic mice, and also reverse adverse effects on axonal transport. In another experimental design, overly dynamic microtubules found in tau-knock out mice contribute to cognitive dysfunction, and these learning deficits are restored by microtubule-stabilizing drugs.

The disclosed method directly stabilizes the microtubule by intrinsically modify the building blocks of microtubules, tubulin. Using rational molecular design, it is possible to create DNA constructs for tubulin that produce more stable, damage-resistant microtubules. Novel tubulin protein sequences (superior stabilizing-tubulins; SS-tubulins) are identified based of the 3-dimensional conformations and physical characteristics of tubulin bound to stabilizing drugs. As further outlined elsewhere in this specification, what degree of enhancement of microtubule stability is desirable, as well as which amino acids need to be considered in order to produce a more stable microtubule within a precisely defined range, such that there is an optimal balance between depolymerization and polymerization of microtubules. Knowing which amino acids promote tubulin-tubulin interactions will directly inform the structural details of gene constructs generated for stabilizing their product, SS-tubulins.

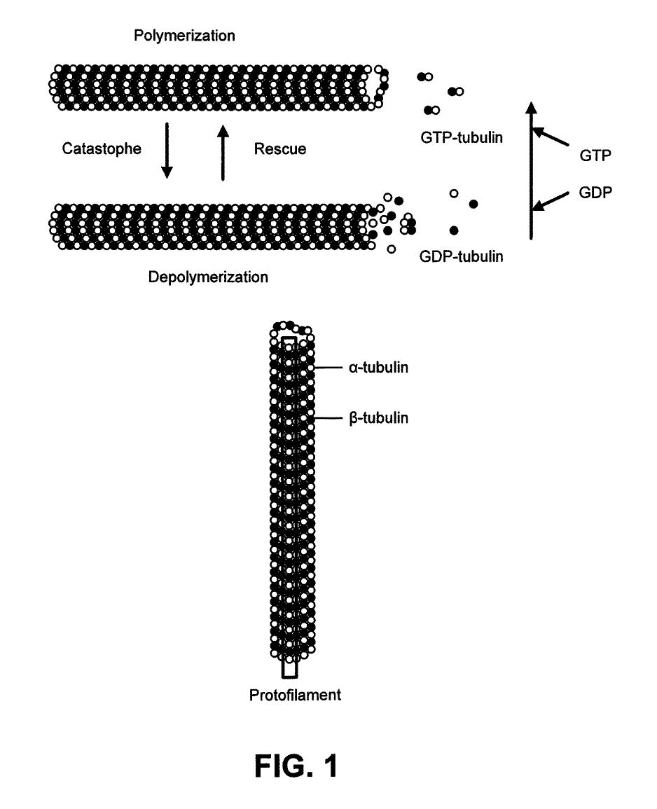

Molecular modeling of tubulin is universally based on the 3-dimensional conformation of the native αβ-tubulin dimer obtained from electron crystallography to a resolution of 3.5 Å. Referring to FIG. 1, each αβ-tubulin dimer assembles longitudinally into microtubule protofilament, which binds through lateral contacts with other protofilaments to form microtubules. In the process of polymerization, tubulin acts as a GTPase, with individual tubulin subunits being added to the growing tip at the expense of GTP hydrolysis. The introduction of specific tubulin thereby gains an opportunity to affect the overall stability of the microtubule. GTP hydrolysis occurs when making lateral bonds occur between the helix H3 region and the M loop of two adjacent tubulins or when longitudinal bonds form between tubulins along the protofilament.

The numbering scheme used in this specification follows the residue numbering convention for 1JFF established by the Protein Data Bank (RCSB PDB) wherein the alpha and beta tubulin were aligned by inserting gaps in THE beta tubulin. This offsets certain numbers (generally by two residues for low numbered sequences or by ten residues for higher numbered sequences). The primary structure for an alpha tubulin (Bos taurus) is given in SEQ ID NO. 1 whereas the primary structure for a beta tubulin (Bos taurus) is given in SEQ ID NO. 2). For example, Glu71 (PDF numbering) is Glu69 (in SEQ ID NO. 2); Asp76 is Asp74; Lys124 is Ly 122; Tyr283 is Tyr281; Arg401 is Arg391; Phe404 is Phe394 and Trp407 is Trp397. Multiple isotypes of both α tubulin and β tubulin are known. Generally, each tubulin isotype comprises fewer than five hundred residues.

TABLE 1
Ten point mutations that increase stability

| Mutation | Interaction | Principle |
|---|---|---|
| αAsp218 to Lys | α-α along lateral interface | αAsp218 is making |
| | | unfavorable electrostatic |
| | | interactions with the |
| | | αGlu90. Replacing either |
| | | with a lysine residue |
| | | reverses this effect |
| αGlu90 to Lys | α-α along lateral interface | αAsp218 is making |
| | | unfavorable electrostatic |
| | | interactions with the |
| | | αGlu90. Replacing either |
| | | with a lysine residue |
| | | reverses this effect |
| αGlu220 to Lys | α-α along lateral interface | The glutamate suffers |
| | | repulsion with βAsp130 |
| | | and αGlu90. Replacing it |
| | | with lystine reverses this |
| | | effect. This mutation may |
| | | not be combined with |
| | | αAsp218 to Lys or αGlu90 |
| | | to Lys. |
| αAla58 to Val | α-α along lateral interface | Strengths the vdW |
| | | interactions made with |
| | | αTyr282. |
| αGlu254 to Gln | α-β along longitudinal | This glutamate is highly |
| αGlu254 to Ala | interface | repulsive to the cofactor at |
| | | the interdimer interface. |
| | | Replacing it stabilizes the |
| | | system. |
| αAsp47 to Asn | α-β along longitudinal | αAsp47 repels βAsp76 |
| | interface | strongly. Replacing either |
| | | of them with an asparagine |
| | | residue establishes a |
| | | hydrogen bond between |
| | | them which also prevents |
| | | other unfavorable |
| | | interactions. |
| αAsp327 to Asn | α-β along longitudinal | This aspartic acid repels |
| | interface | other nearby negatively- |
| | | charged residues. |
| | | Replacing it with |
| | | asparagine establishes a |
| | | hydrogen bond instead. |
| βLys124 to Glu (RCSB | β-β along lateral interface | This lysine is largely |
| PDB numbering) | | destabilizing due to |
| βLys122 to Glu (SEQ ID | | unfavorable electrostatic |
| NO. 2 numbering) | | interactions with the |
| | | mostly positive nearby |
| | | residues. Replacing it with |
| | | glutamate reverses this |
| | | effect. |
| βAsp76 to Asn (RCSB | α-β along longitudinal | αAsp47 repels βAsp76 |
| PDB numbering) | interface | strongly. Replacing either |
| βAsp74 to Asn (SEQ ID | | of them with an asparagine |
| NO. 2 numbering) | | residue establishes a |
| | | hydrogen bond between |
| | | them which also prevents |
| | | other unfavorable |
| | | interactions. |

In most of the above mutations, charged residues with unfavorable electrostatic interactions could rather be replaced with glycine instead of the oppositely-charged residues lest the latter may introduce unexpected interactions.

In some embodiments, one or more of the stability-increasing point mutations are accompanied by one or more stability-decreasing point mutations. Many microtubule functions are optimized by a finely tuned dynamic-stability ratio that falls within a precise range. In certain applications, mutations that increase stabilization are counterbalanced by mutations that decrease stability to optimize the resulting dynamic-stability range.

TABLE 2
Eight point mutations that decrease stability

| Mutation | Interaction | Principle |
|---|---|---|
| βTyr283 to Gly (RCSB | β-β along lateral interface | βTyr283 along provides |
| PDB numbering) | | about 50% of the stability |
| | | at the β-β interface. |
| αHis283 to Gly | α-α along lateral interface | Both histidine 282 and 88 |
| αHis88 to Gly | | provide about 40% of the |
| | | stability at the α-α |
| | | interface. |
| βArg401 to Gly (RCSB | α-β along longitudinal | βArg401, βPhe404 and |
| PDB numbering) | interface | βTrp407 contribute about |
| βPhe404 to Gly(RCSB | | 20% to longitudinal |
| PDB numbering) | | stability with a great deal |
| βTrp407 to Gly(RCSB | | of vdW for the latter two |
| PDB numbering) | | and half vdW half |
| | | electrostatic for the former. |
| βGlu71 to Gly | α-β along longitudinal | These two residues provide |
| αArg2 to Gly | interface | about 10% of the |
| | | longitudinal stability |
| | | mostly due to electrostatic |
| | | interactions. |

There are many advantages to directly modifying protein structure rather than relying on a drug-binding or alternative ligand-binding to produce a particular change in overall protein conformation. These advantages include: (1) reduction of side-effects due to drug toxicity or adverse reaction to immunotherapy, (2) capacity for more selective targeting, and (3) greater permanency of effect (particularly in non-dividing neurons). Once vulnerable cells in AD brain are rescued by the addition of DNA constructs for SS-tubulin, the integrity of the microtubule matrix is fortified. This results in restoration of proper neuron function and transport of materials to pre- and post-synaptic sites. A cell with a healthy cytoskeletal is fully capable of synaptic transmission and cell survival. Possible adverse effects are expected to be minimal, particularly if the therapy is well localized to damaged or vulnerable neurons.

In one embodiment, gene therapy is used to directly alter the intrinsic macromolecular structure by tweaking one or more of the drug target proteins to “naturally” exhibit a 3-dimensional conformation, which would be more similar to the 3-dimensional conformation in the drug-bound state. Structural proteins (as opposed to receptor proteins) that change their 3-dimensional conformation may be the ideal test because maintaining stability (within a certain allowable dynamic) is one of their fundamental roles.

The method directly alters the intrinsic macromolecular structure by tweaking one or more of the drug target proteins to “naturally” exhibit a 3-dimensional conformation, which would be more similar to the 3-dimensional conformation in the drug-bound state. Structural proteins (as opposed to receptor proteins) that change their 3-dimensional conformation may be the ideal initial test because maintaining stability (within a certain allowable dynamic) is one of their fundamental roles. Without wishing to be bound to any particular theory, introducing SS-tubulin and/or SS-tubulin DNA into damaged brain areas is believed to confer greater structural stability to microtubules and thereby ameliorate the root cause of many cellular dysfunctions that arise in AD diseased cells.

Knowing how to regulate microtubule dynamics is fundamental to preventing cellular degeneration. Understanding how stabilizing drugs achieve this end provides one path. When tubulin is bound to various stabilizing drugs (e.g., paclitaxel, epothilone, zampanolide) its overall structural conformation changes slightly but significantly enough to enable increased binding to the neighboring tubulin dimers. The net effect stabilizes microtubules and enhances polymerization. A related approach is to modify tubulin to enhance lateral and longitudinal bonds with neighboring tubulins. Described herein is a method of using rational molecular design to produce superior stabilizing-tubulin molecules (SS-tubulin).

In one embodiment, gene therapy is used to deliver SS-tubulin DNA that encodes for SS-tubulin. In other embodiments other nanotech delivery systems are used. The SS-tubulin DNA may be delivered alone or in conjunction with current gene therapies for AD. As an example, SS-tubulin DNA could be added to gene therapies that increase neurotrophins in damaged regions of AD patients. In this scenario, the added SS-tubulin DNA would facilitate retrograde transport of the neurotrophin to the cell body, enabling the neurotrophin to exert its full effects.

This capability of gene therapy is believed to enhance microtubule stability is exactly what is necessary to produce clinical improvement in memory. Proper microtubule stabilization will not only ameliorate neuroplasticity deficits, it will repair anterograde and retrograde transport tracks, thereby restoring neurotransmitter and neurotrophin transport to their effective sites in the neuron. Time and time again, drugs which prove promising in preclinical trials remain largely ineffective because the internal structural matrix needed for responding to those chemical messengers is compromised in AD brain.

There is foreseeable promise in using gene therapy for SS-tubulin to revitalize those vulnerable neuron populations in AD (i.e., neurons in hippocampus, amygdala, select cortical regions, and basal forebrain). Repairing the internal machinery underlying neuroplasticity addresses most, if not all, issues attendant with AD, including cognitive decline, neuropathology accumulation, neurotransmission failure, and cell degeneration. In the near-term, gene therapy for SS-tubulin might be tried as an adjunct to existing gene therapies, for example, added to gene therapy for NFG. Many researchers have noted that NGF therapy would undoubtedly fare better if axonal transport could be improved.

Creating DNA constructs for SS-tubulin and applying gene therapy protocols used in ongoing clinical trials for AD represents just one means of stabilizing microtubules to achieve cognitive improvement, reduce neuropathology, prevent degeneration, and restore connectivity. Nanomedicine offers up a host of nanoparticles and nanocarriers as novel ways to carry molecules into the AD brain. There are additional nanotech methods to bioengineer biohybrid or synthetic materials to make stronger, more stable microtubules or bionic alternatives. As an exciting new field, nanotech provides the necessary tools and the engineering perspective needed to fix broken transport systems in AD, perhaps far better than neuropharmacology because its emphasis is on the biomechanical aspects of cell components. A paradigm shift towards fixing structural defects in neurons is a significant departure from the status quo. Nanomedicine is perhaps just the fresh outlook needed to tackle neurodegenerative disorders like AD, hopefully producing the same kind of exponential advances that occurred over 60 years ago for neuropharmacology when several drugs simultaneously rose to the forefront and modernized treatment of psychiatric and neurological disorders.

Nanocarriers and nanoparticles already developed are capable of delivering SS-tubulin DNA constructs to specific brain regions, and there are nanotech approaches to selectively target degenerating cells. Some nanoparticles and nanocarriers have magnetic properties, such that sufficiently strong and well-focused magnetic fields can be applied from an external source to guide the attached cargo to the desired brain site. A magnetic nanocarrier has already succeeded in carrying brain-derived neurotrophic factor (BDNF) across the blood-brain-barrier and effectively halting cell death and degeneration in a substance-abuse animal model. These kinds of approaches used in conjunction with SS-tubulin gene therapy stand to greatly enhance effectiveness, simplify administration, and eventually reduce cost of microtubule-stabilizing therapy.

A related approach is to reinforce microtubule stability directly, in conjunction with SS-tubulin therapy or alone. Preliminary data are supportive. Magnetic nanoparticles conjugated to microtubule-signaling proteins enable researchers to control microtubule function by applying an external magnetic field. In this manner, microtubule assembly can be switched on and off. Microtubules coated with metals (such as silver, gold, and cobalt ferrite) also can be guided by externally generated electrical or magnetic fields. In one possible scenario, metallized microtubules or metallized nanocarriers carrying SS-tubulin DNA constructs could be introduced into the blood circulation, much the same as is done before certain brain imaging techniques, after which external magnetic or electrical fields could be applied to the patient directing the cargos to a particular brain region. Nanocarriers also could be pulsed along intrinsic microtubules, since microtubules in vitro respond to electrical fields.

The future of therapeutic measures for degenerative disorders like AD will likely see significant advances, particularly with approaches that directly target microtubule function. Microtubule deficits are amendable to a vast array of nanotech approaches that will ultimately provide a high-precision and low-cost means to restore failing intracellular functions to a healthy state. Various nanotech approaches to stabilize microtubule function are outlined below.

In one embodiment, gene therapy is used to enhance microtubule stability. Microtubules are assemblies of tubulin protein subunits, of which over 400 sequences are known. Variations in tubulin structure (as determined by amino acid sequence) contribute to greater stability versus instability based on effects on polymerization-depolymerization rate (regulated by tubulin-to-tubulin binding). Genes for superior stabilizing-tubulin (SS-tubulin) are added to enhance microtubule stability, facilitate neurotransmitter reception, and axonal transport. Gene therapies may be used to introduce viral vectors into select brain regions for treating Alzheimer's and Parkinson's patients. A key obstacle in this research is that transport (which is a microtubule function) is faulty causing reduced efficacy of the genetically introduced neurotrophins. By adding genes for select tubulin proteins that stabilize the microtubule and facilitate transport to those existing protocols, outcomes would be enhanced because higher levels of neurotrophins would reach their targets. Tubulin gene therapy exerts its own independent benefit, as well. Advantages include introducing genes for select tubulins into neurons will produce a permanent change, since neurons are long-lived. The technology platform is already developed with clinical trials in progress. Approval for adding genes to increase microtubule stability to an on-going project would be faster than obtaining approval as a separate project. Potential drawbacks include the use of neurosurgery for introducing gene vectors which is highly invasive, expensive, and poses risk to patients. While unlikely to become a widely used solution for degenerative disease, it provides a feasible way to access cells to halt degeneration while the scientific community develops nanotech approaches that would accomplish the same end.

In one embodiment, nanocarriers and nanoparticles is used to enhance microtubule stability. Genes for SS-tubulin could be delivered to enhance microtubule stability, facilitate neurotransmitter reception, and axonal transport. Many nanocarriers and nanoparticle have been tested in animal models. Advantages include Nanocarriers and nanoparticles to deliver drugs or genes to brain cross the blood bran barrier and circumvent the need for neurosurgery in the case of gene therapy. Selective targeting of specific brain areas is achieved with magnetic fields from an external source after magnetic nanocarriers or nanoparticles are delivered. Potential drawbacks include potential side effects are still unknown.

In one embodiment, nanocapsules are used to enhance microtubule stability. Nanocapsules attached to cantilevers detecting markers for degeneration open to allow for the release of mRNA that encodes for tubulin proteins or drugs that confer stability to the cell or facilitate transport and cell survival. Advantages include this approach would be minimally invasive and effective at targeting a precise structure.

In one embodiment, hybridized microtubule-nanoparticles are used to enhance microtubule stability. Small microtubule segments bound to metal or components in nanocapsules are ingested {or initially injected into the carotid artery) and carried to the brain where they are positioned in the target structures using magnetic fields. Advantages include this approach would be minimally invasive (or non-invasive through oral administration) and effective at targeting a precise structure. Because microtubules turn over within hours to days, the therapy would have to be repeatedly administered or continuously delivered.

A method is provided to design novel DNA sequences for SS-tubulin, representing unique 3-dimensional conformations of tubulin, which make slightly stronger bonds with adjacent tubulins. These alterations are based on a wealth of information deriving from homology modeling, paying particular attention to the subtle differences in physical properties produced by slight alterations to the tubulin protein sequence.

Previous studies applied homology modeling to reveal the overall structure and the physical properties (e.g., net electrical charge, solvent accessible surface areas, dipole moments) of well over 400 sequences of tubulin. This includes the 15 human tubulins, other animal and plant tubulins, and encompasses examples of α-tubulin, β-tubulin, γ-tubulin, δ-tubulin, and ε-tubulin isotypes. Structural and physical characteristics of each unique tubulin sequence determine the binding properties of that tubulin molecule and its ability to strongly impact overall microtubules stability (i.e., polymerization/depolymerization).

Sequences for novel SS-tubulin constructs can be based on best fit to the overall 3-dimensional conformation of tubulin bound to stabilizing drugs (i.e., drugs that alter tubulin structure in favor of enhancing overall microtubule stability). Drugs that bind microtubules and enhance its stability include paclitaxel, epothilone, and zampanolide. Although these different drugs bind to slightly different sites, a common action is stabilization of the M loop of tubulin, a site that depending on its conformational status can influence lateral bonding between tubulins. The 3-dimensional conformational changes in tubulin structure in the drug-bound state contribute to microtubule stability, but this varies somewhat for different isotypes of tubulin. Paclitaxel, for example, binds to different tubulin isotypes slightly differently: binding to one site on the βIII-tubulin and to six sites on βVI-tubulin.

To produce a more optimal 3-dimensional conformation of tubulin, amino acid substitutions can be placed near or distant to drug-binding sites, but not at the actual sites of tubulin-tubulin contacts. It is already well established that point mutations in tubulin, which arise when cells acquire resistance to anti-cancer drugs such as paclitaxel and epothilone, affect the stability of microtubules. Accordingly, it is possible to manipulate sites mediating a tubulin molecule's ability to stabilize microtubules by substituting amino acids located in and around the M loop, the GTP-binding site, and the sites where MAPs bind.

Post-translational modifications to tubulin are crucial to consider because these also affect the stability of microtubules. Most, but not all, of these sites are located on the C-terminus of tubulin and include: tyrosination (or detyrosination), deglutamylation, polyglutamylation, acetylation, phosphorylation, and polyglycylation. Posttranslational modifications can affect microtubule stability, as illustrated by a specific loss of acetylated tubulin in AD correlated with decreased microtubule stability. Because of their impact, one must consider potential posttranslational modifications when selecting amino acid substitutions for SS-tubulin DNA constructs so that any possible posttranslational modification would effectively alter the stability of microtubules in the intended direction.

One must also considered the effects of stabilizing MAPs, most notably tau, which are critical to the assembly and maintenance of microtubules in neurons and have a stabilizing influence on the microtubule. The 3-dimensional conformation of tubulin changes when MAPs bind. The binding site of tau lies near helices 11 and 12 of α-tubulin, and when tau is bound, tubulins in microtubules strengthen their lateral bonds. Other stabilizing MAPs bind at a similar site near helices 11 and 12, and in the case of binding MAP2c, microtubules further strengthen longitudinal bonds, and this in turn decreases overall flexibility and thereby increases their stability. The increase in stability caused by paclitaxel is accompanied by the opposite, an increase in microtubule flexibility. Thus, one must weight flexibility less as compared to other factors.

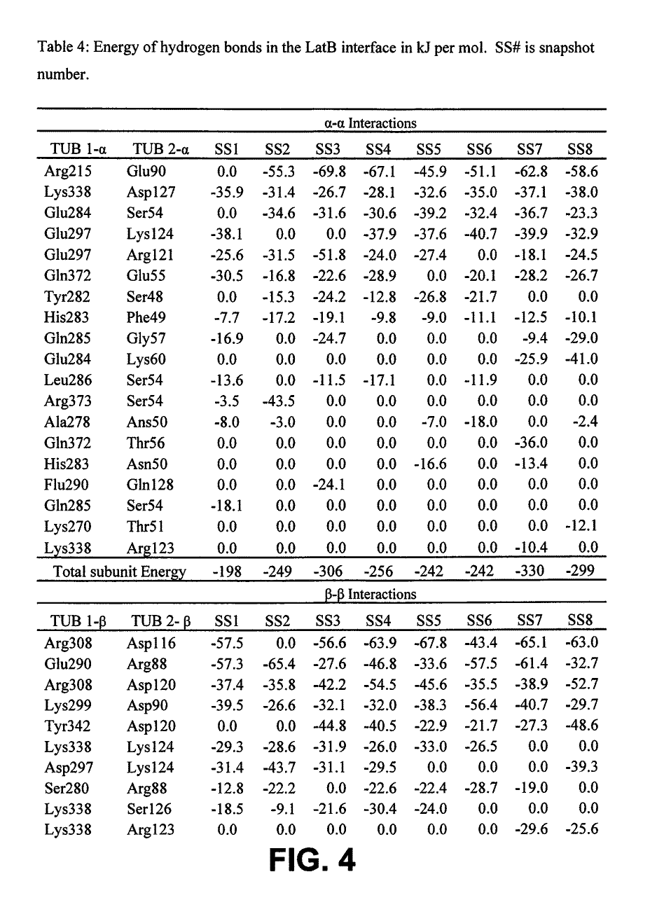

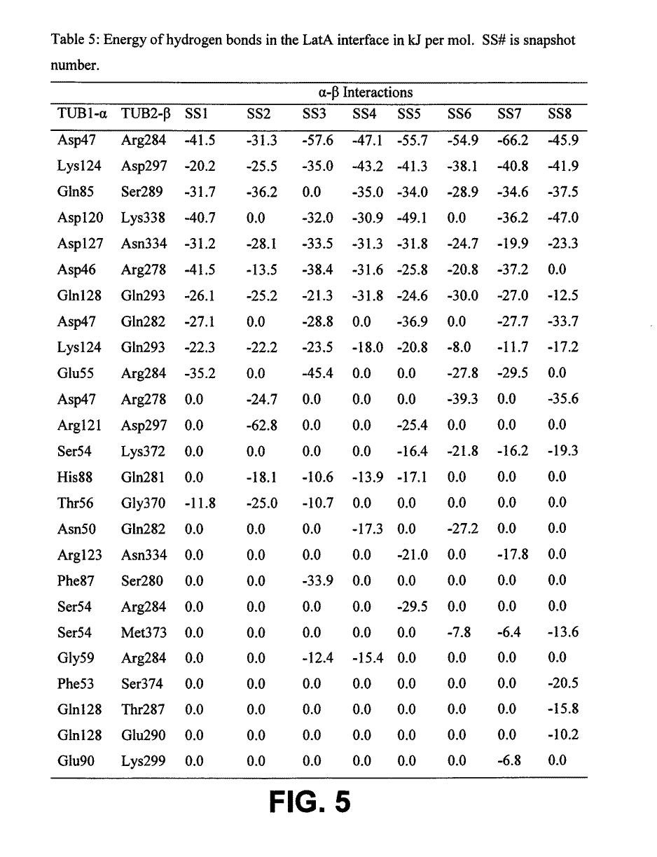

The method places particularly strong emphasis on hydrogen bonds between tubulins when designing SS-tubulin because an extensive interfacial hydrogen-bonding network is largely responsible for tubulin dimers binding to form intact microtubules, and the energetics of each bond contributes proportionally (Table 3 (FIG. 3), Table 4 (FIG. 4) and Table 5 (FIG. 5)). Based on recent modeling, the hydrogen-bonds between tubulins within a protofilament (longitudinal bonds) and tubulin-tubulin contacts between protofilaments (lateral bonds) contribute markedly to the overall stability of microtubules. Residues near the colchicine-binding site strongly regulate longitudinal microtubule stabilization. Lateral contacts showed even higher stability than longitudinal contacts, suggesting a dramatic lateral stabilization effect of the GTP cap in the β-tubulin. In the absence of paclitaxel, the M-loop plays less of a role in lateral stability than expected. With respect to the overall conformation of the microtubule, the B-lattice lateral hydrogen bonds are comparable in strength to the A-lattice.

A Step-by-Step Procedure for Generating SS-Tubulin DNA Sequences

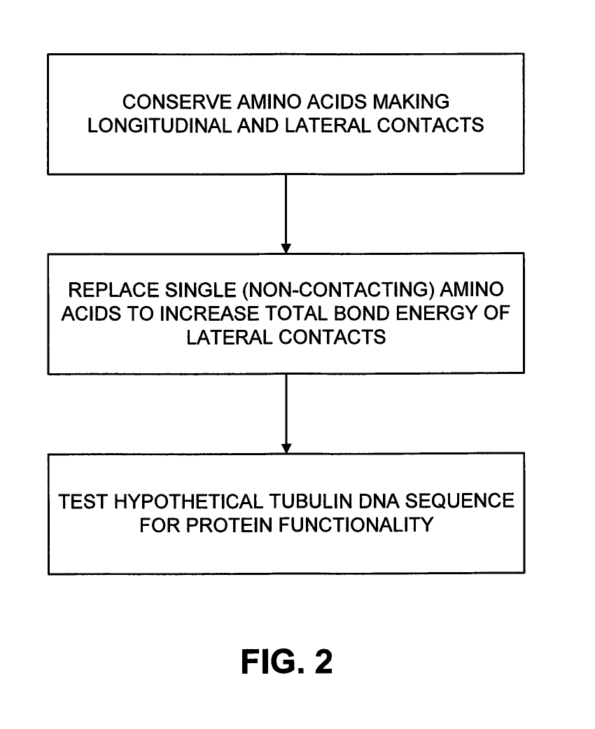

The bond energies for longitudinal and lateral contacts are used as an end-point to predict novel tubulin DNA sequences that will confer superior stability (i.e. SS-tubulin) when assembled into microtubules. As shown in FIG. 2, rational molecular modeling programs can generate thousands of novel tubulin DNA sequences using as a starting point DNA sequences for healthy human tubulins. Keeping the key amino acids that participate in longitudinal or lateral bonds constant, one can compute the bond energies with successive single (or multiple) point substitutions at key loci in tubulin, solving for an increase in overall bond energies due to potential shifts in the positioning of those key amino acids. Three-dimensional conformation shifts caused by amino acid substitutions at other sites in the protein are known to affect lateral binding between tubulins on adjacent protofilaments. The M loop, for example, which does not appear to contribute to lateral bonds when unbound, has influence on lateral bonding with neighboring tubulins when that site is occupied (by paclitaxel, for example) even though the M loop is not a tubulin-tubulin binding site. Following the identification of candidate sequences, filtering out non-functional sequences would result in potentially viable SS-tubulin DNA sequences for further testing in a biological assay (i.e. for effects on polymerization, stability or resistance to depolymerization, function ability to transport cargo, etc.). Using this strategy ten point mutations were identified that increase microtubule stability and eight point mutations that decrease microtubule stability. See Table 1 and Table 2.

The focus of this method is on treating AD; however, a diverse selection of other diseases—neurodegenerative, neurodevelopmental, neuropsychiatric, cardiac, and cancer—involve some degree of microtubule dysfunction. These diseases, disorders, and conditions might be similarly improved by microtubule stabilization. Examples include neurodegenerative diseases and brain injury (Alzheimer's disease, Parkinson's disease, Huntington's chorea, epilepsy, amyotrophic lateral sclerosis, Charcot-Marie-Tooth disease, multiple sclerosis, stroke and brain injury); Neurodevelopmental disorders (fragile X syndrome, Turner syndrome, Williams syndrome, Autism spectrum, Rett syndrome, Down syndrome); Neuropsychiatric disorders (schizophrenia, bipolar disorder depression and anxiety, alcohol and substance abuse); heart disease (congestive heart failure, cardiac arrest, heart transplant and heart valve replacement, congenital heart disease) and cancer (breast cancer, lung cancer, prostate cancer, brain cancer).

The strategy outlined in this disclosure for generating novel DNA constructs that produce tubulins capable of assembling into structurally stabilized microtubules can be applied to other cellular proteins. The fundamental principle is to use genetic engineering to produce “pseudo-drug bound protein” that are essentially analogs with 3-dimensional conformations similar to that of the native protein when bound to an exogenous or endogenous ligand that produces a desirable cellular effect. The net effect is to shift protein function towards the healthy state and away from the disease state (for tubulin this translates to favoring stability favored over instability in assembled microtubules). According to a step-by-step process, this can be achieved this through reverse homology modeling, wherein protein sequences having a greater than 90% (or in another application greater than 80%) structural similarity to the drug-bound state are identified as candidates for further testing.

This written description uses examples to disclose the invention, including the best mode, and also to enable any person skilled in the art to practice the invention, including making and using any devices or systems and performing any incorporated methods. The patentable scope of the invention is defined by the claims, and may

What is claimed is:

1. A method of treating a patient to provide a therapeutic benefit for Alzheimer's Disease (AD), the method comprising administering to a human patient at least an alpha tubulin that is at least 70% homologous with SEQ ID NO. 3, wherein at least one point mutation is present, the at least one point mutation being selected from the group consisting of Asn47; Val58; Lys90; Lys218; Lys220; Gln254 or Ala254; Asn327 and combinations thereof.

2. The method as recited in claim 1, wherein at least two of the point mutations are present.

3. The method as recited in claim 1, wherein at least three of the point mutations are present.

4. The method as recited in claim 1, wherein at least four of the point mutations are present.

5. The method as recited in claim 1, wherein at least five of the point mutations are present.

6. The method as recited in claim 1, wherein at least six of the point mutations are present.

7. The method as recited in claim 1, wherein all seven of the point mutations are present.

8. The method as recited in claim 1, wherein residue 254 is Gln254.

9. The method as recited in claim 1, wherein residue 254 is Ala254.

10. The method as recited in claim 1, wherein the alpha tubulin has fewer than five hundred residues and is at least 80% homologous with SEQ ID NO. 3.

11. The method as recited in claim 1, wherein the alpha tubulin is at least 90% homologous with SEQ ID NO. 3.

12. The method as recited in claim 1, wherein the alpha tubulin is at least 95% homologous with SEQ ID NO. 3.

13. The method as recited in claim 1, wherein the alpha tubulin is administered by introducing a gene that encodes for the alpha tubulin to the human patient.

14. A method of treating a human patient to provide a therapeutic benefit, the method comprising administering at least a beta tubulin that is at least 70% homologous with SEQ ID NO. 4, wherein at least one point mutation is present, the at least one point mutation being selected from the group consisting of Glu122; Asn74 and combinations thereof.

15. The method as recited in claim 14, wherein all two of the point mutations are present such that residue 122 is Glu122 and residue 74 is Asn74.

16. The method as recited in claim 14, wherein the beta tubulin has fewer than five hundred residues and is at least 80% homologous with SEQ ID NO. 4.

17. The method as recited in claim 14, wherein the beta tubulin is at least 90% homologous with SEQ ID NO. 4.

18. The method as recited in claim 14, wherein the beta tubulin is at least 95% homologous with SEQ ID NO. 4.

19. The method as recited in claim 14, wherein the beta tubulin is administered by introducing a gene that encodes for the beta tubulin to the human patient.


SEQUENCE LISTING (2 sequences)

SEQ ID NO: 1 - protein, 452 aa, Bos taurus
MRECISVHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKELIDLVLDRIRKLADQCTGLQGFLIFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGMDSVEGEGEEEEGDEY

SEQ ID NO: 2 - protein, 445 aa, Bos taurus
MREIVHLQAGQCGNQIGAKFWEVISDEHGIDPTGTYHGDSDLQLERINVYYNEATGGKYVPRAVLVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQMFDAKNMMAACDPRHGRYLTVAAVFRGRMSMKEVDEQMLNVQNKNSSYFVEWIPNNVKTAVCDIPPRGLKMSATFIGNSTAIQELFKRISEQFTAMFRRKAFLHWYTGEGMDEMEFTEAESNMNDLVSEYQQYQDATAEEEGEFEEEAEEEVA